(19) United States
(12) Patent Application Publication
(10) Pub. No.: US 2007/0100171 A1
(43) Pub. Date: May 3, 2007

(54) Method of detecting decabromodiphenyl ether

(75) Inventor: Kanako Ogawa, Hino-shi (JP)

Correspondence Address: PILLSBURY WINTHROP SHAW PITTMAN, LLP, P.O. BOX 10500, MCLEAN, VA, 22102, US

(73) Assignee: KABUSHIKI KAISHA TOSHIBA, Tokyo (JP)

(21) Appl. No.: 11/585,187

(22) Filed: Oct. 24, 2006

(30) Foreign Application Priority Data
Oct. 31, 2005 (JP) 2005-316383

(51) Int. Cl. C07C 43/02; C07C 43/20

(52) U.S. Cl. 568/661

(57) ABSTRACT

A method of detecting decabromodiphenyl ether, includes applying tetrahydrofuran to a sample, and then adding n-hexane thereto, drying the supernatant of the resultant, and subjecting the supernatant to infrared spectrometry.

### CROSS-REFERENCE TO RELATED APPLICATIONS

This application is based upon and claims the benefit of priority from Japanese Patent Application No. 2005-316383, filed Oct. 31, 2005, the entire contents of which are incorporated herein by reference.

### BACKGROUND

1. Field

The present invention relates to a method of detecting decabromodiphenyl ether, which is added as a bromine-based flame retardant to a flammable substance of parts such as of plastic, rubber and woven fabric.

2. Description of the Related Art

Flammable materials such as resin parts of plastic and rubber used in electronic or electric devices contain, for example, a bromine-based flame retardant added thereto for lowering the combustion speed thereof or suppressing the combustion. A typical example of the bromine-based flame retardant is decabromodiphenyl ether, which is a type of polybrominated phenyl ether.

Decabromodiphenyl ether is a substance that is regulated under eco-labels such as Blue Engel Mark (BEM) of Europe, TCO-95 and White Swan or Electronic Device Waste Order. Therefore, it is necessary to select out resin parts that contain decabromodiphenyl ether from the parts taken from spent electronic devices.

For example, Jpn. Pat. Appln. KOKAI Publication No. 5-60705 discloses a technique of analyzing bromine-based flame retardants added to resin materials by extracting them using a solvent and then subjecting them to infrared spectrometry.

However, in order to prepare a sample to be analyzed, the above-mentioned spectrometric method requires a step of concentrating a solution of an extract taken from a part using a solvent to a concentration that can be analyzed, and the concentration step is complicated and time-consuming.

Under these circumstances, there has been a demand for a method of easily checking the presence/absence of decabromodiphenyl ether in a resin.

### BRIEF DESCRIPTION OF THE SEVERAL VIEWS OF THE DRAWINGS

A general architecture that implements the various feature of the invention will now be described with reference to the drawings. The drawings and the associated descriptions are provided to illustrate embodiments of the invention and not to limit the scope of the invention.

FIG. 1 is a flow diagram illustrating an example of the method of detecting decabromodiphenyl ether, according to the present invention;

FIG. 2 is a graph indicating spectral data used in an example of detection of decabromodiphenyl ether; and

FIG. 3 is a graph indicating spectral data used in another example of detection of decabromodiphenyl ether.

### DETAILED DESCRIPTION

Various embodiments according to the invention will be described hereinafter with reference to the accompanying drawings. In general, according to one embodiment of the invention, tetrahydrofuran is used as a solvent to prepare a sample solution. Tetrahydrofuran can dissolve various types of resins, and further it can elute decabromodiphenyl ether from a sample. From an obtained sample solution, a partial amount of supernatant is taken, and dried. Then, the dried supernatant is subjected to the infrared spectrometry to analyze a content of the supernatant. Thus decabromodiphenyl ether can be detected.

With the method according to the embodiment of the present invention, it is possible to obtain a sample to be analyzed in the infrared spectrometry simply by applying tetrahydrofuran to the sample. The preparation of the sample to be analysis here requires no step of concentration or purification after applying the solvent. Thus, complicated procedures or devices are not required, and the operation is easy. Further, the preparation of the sample can be carried out at ordinary room temperature or further at ordinary room temperature and ordinary pressure. The sample to be analyzed used in the embodiment of the present invention may be difficult to handle in an accurate quantitative analysis of decabromodiphenyl ether; however, it is sufficient to check the presence/absence of decabromodiphenyl ether, which can be carried out in a simple manner at a low cost.

According to the method of detecting decabromodiphenyl ether according to the other embodiment of the present invention, tetrahydrofuran is used as a solvent to prepare a sample solution. The first sample solution is prepared and a partial amount of the supernatant is taken therefrom. After that, using n-hexane, the second sample solution is prepared. Furthermore, a partial amount of the supernatant is taken from the obtained second sample solution, and dried. Then, the resultant is subjected to the infrared spectrometry to analyze a content of the supernatant.

Tetrahydrofuran can dissolve various types of resins, and further it can elute decabromodiphenyl ether from a sample. However, in some cases, depending on the type of resin used as a sample, it has a peak analogous to that of the infrared spectrum of decabromodiphenyl ether. As a result, it is not possible in such cases to identify the peak of decabromodiphenyl ether as the peaks of both substances superimposed on each other. In order to deal with such a case, n-hexane is added to a partial amount of the supernatant that contains tetrahydrofuran to precipitate the resin used in the sample, and thus the second sample solution is prepared. From the second sample solution, a partial amount of the supernatant thereof is dispensed and dried, and the resultant is subjected to the infrared spectrometry.

With the method according to the other embodiment of the present invention, it is possible to obtain a sample to be analyzed in the infrared spectrometry simply by applying tetrahydrofuran and n-hexane to the sample. The preparation of the sample to be analysis here requires no step of concentration or purification after applying the solvent. Thus, complicated procedures or devices are not required, and the operation is easy. Further, the preparation of the sample can be carried out at ordinary room temperature or further at ordinary room temperature and ordinary pressure. The sample to be analyzed used in the present invention may be difficult to handle in an accurate quantitative analysis of decabromodiphenyl ether; however, it is sufficient to check the presence/absence of decabromodiphenyl ether, which can be carried out in a simple manner at a low cost. Further, even in case where a rein that has a peak analogous to that of the infrared spectrum of decabromodiphenyl ether is employed as a sample, such a resin can be precipitated by adding n-hexane, which does not easily dissolve various types of resins. In this manner, the resin that interferes with the detection of decabromodiphenyl ether cannot easily mix into the supernatant. Therefore, it is possible to obtain such an infrared spectrum that can easily identify decabromodiphenyl ether, and therefore the detection of the substance becomes easy.

The present invention will now be described in further detail with reference to accompanying drawings.

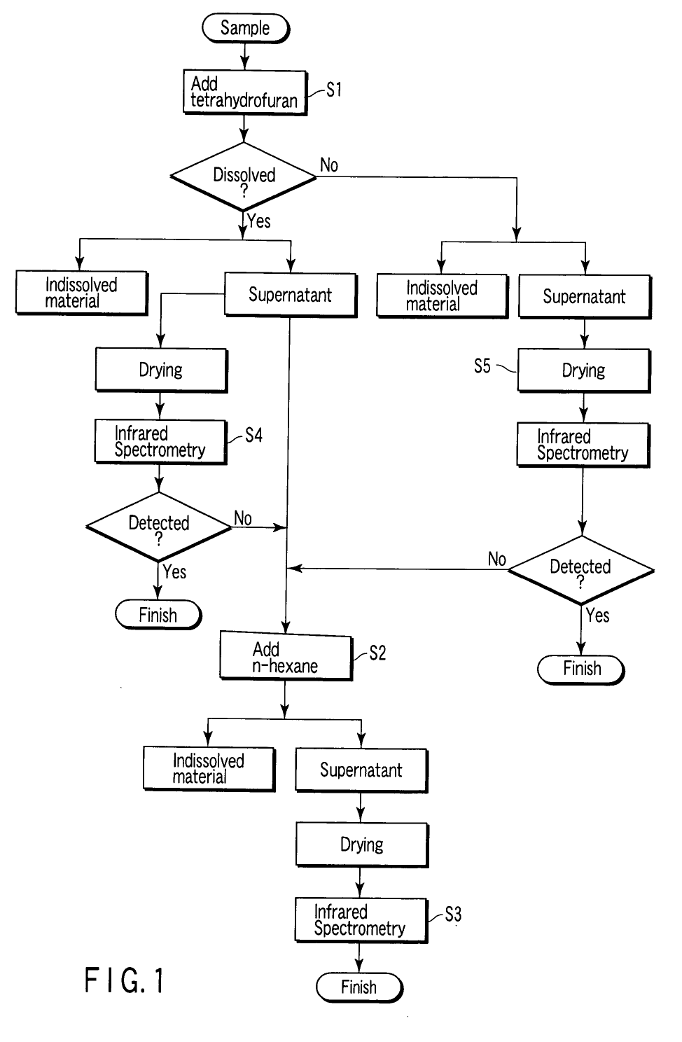

FIG. 1 is a flow chart illustrating an example of the method of detecting decabromodiphenyl ether, according to the present invention.

As indicated in the chart, first, a sufficient amount of tetrahydrofuran is added to a sample to prepare the first sample solution to extract (S1).

In the case where the sample is dissolved out, n-hexane is added to the supernatant of the first sample solution to prepare the second sample solution, and thus the resin component of the dissolved sample is precipitated (S2).

Further, the supernatant is dispensed from the second sample solution and dried. Then, the resultant is subjected to the infrared spectrometry (S3). The presence/absence of decabromodiphenyl ether is judged from the obtained results and thus the detection is finished.

Here, it is alternatively possible to, before adding n-hexane to the supernatant of the first sample solution, dispense the supernatant arbitrarily, and dry the resultant, which is to be subjected to the infrared spectrometry (S4). In the case where decabromodiphenyl ether is detected from the obtained results at this stage, it is judged that the sample contains decabromodiphenyl ether, and the detection is finished. On the other hand, in the case where decabromodiphenyl ether is not detected, the above-described steps S2 and S3 are carried out, and the presence/absence of decabromodiphenyl ether is judged from the obtained results. Thus, the detection is finished.

When the sample is not dissolved, the supernatant is dispensed and dried, and the resultant is analyzed by the infrared spectrometry (S5). In the case where decabromodiphenyl ether is detected from the obtained results at this stage, it is judged that the sample contains decabromodiphenyl ether, and the detection is finished. On the other hand, in the case where decabromodiphenyl ether is not detected, the above-described steps S2 and S3 are carried out, and the presence/absence of decabromodiphenyl ether is judged from the obtained results. Thus, the detection is finished.

### EXAMPLES

The present invention will now be described in further detail by presenting Examples.

### Example 1

First, an acrylonitrile-butadien-styrene resin containing 1 to several % by weight of decabromodiphenyl ether was prepared as a sample.

A portion of several tens to 100 mg was taken from the sample, and put in a vial.

About 1 ml of tetrahydrofuran was added to the vial to prepare the first sample solution. The sample was immersed sufficiently and let stand about half a day to extract the target. After being let stand, the sample was dissolved substantially completely.

0.5 ml of the supernatant of the first sample solution was dispensed and put in another vial. Then, about 0.5 ml of n-hexane was added to prepare the second sample solution, in which precipitation occurred.

About 10 to several tens micro-liters of the supernatant of the second sample solution was dropped on a metal plate, and thus the supernatant was dried.

Thus obtained residual was analyzed with a microscopic infrared spectrometric device, and thus infrared spectral data were obtained.

Thus obtained infrared spectral data were compared with standard spectral data of decabromodiphenyl ether, and the result indicated that the obtained data substantially match with the absorption wavelength of decabromodiphenyl ether. Thus, it was judged that the sample contains decabromodiphenyl ether and thus the detection was finished.

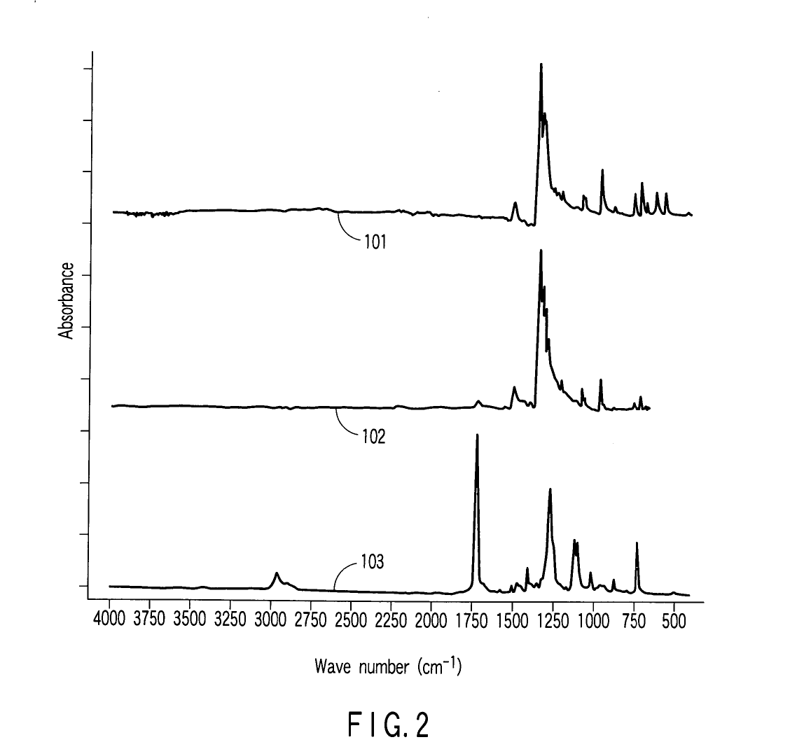

FIG. 2 shows the spectral data used in the detection of decabromodiphenyl ether. This figure shows a graph 101 indicating the standard spectral data of decabromodiphenyl ether, a graph 102 indicating the infrared spectral data obtained by using the supernatant of the second sample solution, and a graph 103 indicating the infrared spectral data of the sample.

As can be seen in the figure, the graph 103 of the infrared spectral data of the sample resin showed an absorption wavelength peak that matches with that of the standard spectral data of decabromodiphenyl ether. Although it is not possible to confirm the presence/absence of decabromodiphenyl ether from the graph 103, the graph 102 of the infrared spectral data for the supernatant of the second sample solution obtained by the method of the present invention showed an absorption wavelength peak that substantially matches that of the graph 101 of the standard spectral data of decabromodiphenyl ether.

Apart from the above, about 10 to several tens micro-liters of the supernatant of the first sample solution was dropped on a metal plate, and thus the supernatant was dried.

Thus obtained residual was analyzed with the microscopic infrared spectrometric device, and thus infrared spectral data were obtained.

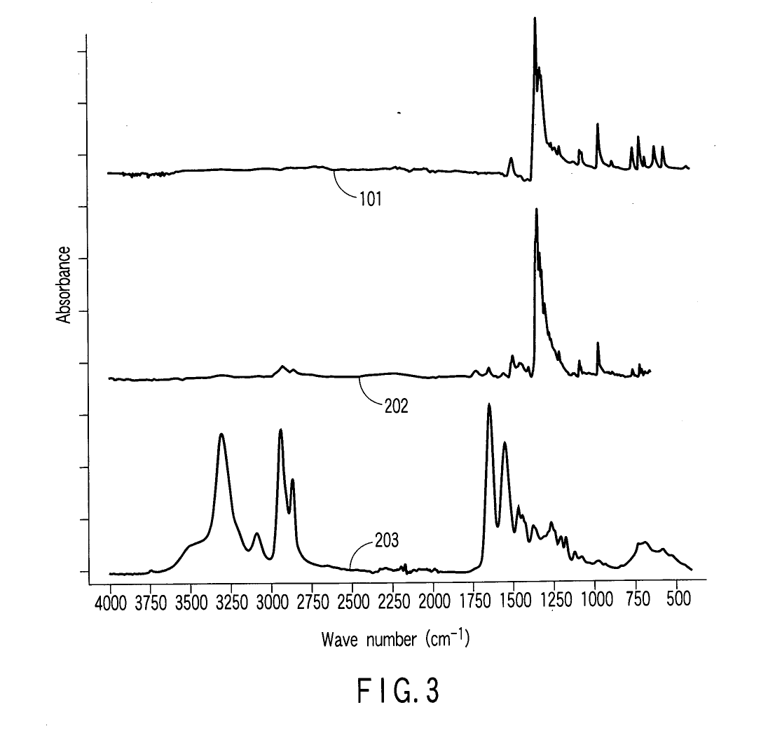

Thus obtained infrared spectral data were shown in FIG. 3.

This figure shows a graph 101 indicating the standard spectral data of decabromodiphenyl ether, a graph 202 indicating the infrared spectral data obtained by using the supernatant of the first sample solution, and a graph 203 indicating the infrared spectral data of the sample.

As can be seen in the figure, the graph 202 of the infrared spectral data showed an absorption wavelength peak that substantially matches with that of the graph 101 of the standard spectral data of decabromodiphenyl ether. Thus, it was found that the detection of decabromodiphenyl ether can be carried out by the infrared spectrometry of only the first sample solution.

It should be noted here that the preparation of the first and second sample solution was carried out at a ordinary room temperature and ordinary pressure.

### Examples 2 to 6

The detection of the target substance was carried out in a similar manner to that Example 1 except that the resin used as the sample was changed to polyester, polyethylene, polycarbonate, nylon or polypropylene, respectively.

Of the above-mentioned resins, polyester, polycarbonate and polypropylene each showed a absorption wavelength peak that matches with that of the standard spectral data of decabromodiphenyl ether.

Further, in connection with polyester, polyethylene, nylon and polypropylene, tetrahydrofuran was added to prepare the first sample solution, which was let stand for half a day, but the sample was not substantially dissolved in each case. Therefore, polyester, polyethylene, nylon and polypropylene can be subjected to the detection of decabromodiphenyl ether by the infrared spectrometry of only the first sample solution.

While certain embodiments of the inventions have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

What is claimed is:

1. A method of detecting decabromodiphenyl ether, comprising:
preparing a sample solution by applying tetrahydrofuran to a sample; and
dispensing a partial amount of supernatant from the sample solution and drying to analyze a content of the supernatant by infrared spectrometry.

2. The method according to claim 1, wherein the preparing of the sample solution is carried out at a ordinary room temperature.

3. A method of detecting decabromodiphenyl ether, comprising:
preparing a first sample solution by applying tetrahydrofuran to a sample;
dispensing a partial amount of supernatant of the first sample solution;
preparing a second sample solution by applying n-hexane to the partial amount of supernatant; and
dispensing a partial amount of supernatant from the second sample solution and drying to analyze a content of the supernatant by infrared spectrometry.

4. The method according to claim 3, further comprising dispensing a partial amount of supernatant from the second sample solution before adding n-hexane to the partial amount of supernatant, and then drying to analyze a content of the supernatant of the first sample solution by infrared spectrometry.

5. The method according to claim 3, wherein the preparing of the first sample solution and the second sample solution is carried out at a ordinary room temperature.
